(19) United States
(12) Patent Application Publication
(10) Pub. No.: US 2013/0046221 A1
(43) Pub. Date: Feb. 21, 2013

(54) Heel support brace

(71) Applicant: Robert S. Christman, Ione, CA (US)

(72) Inventor: Robert S. Christman, Ione, CA (US)

(73) Assignee: Robert Stephen Christman, Ione, CA (US)

(21) Appl. No.: 13/199,054

(22) Filed: Aug. 16, 2011

(51) Int. Cl. A61F 5/00

(52) U.S. Cl. 602/28

(57) ABSTRACT

This invention is a heel support brace. This heel support brace is a solid neoprem/nylon type of brace with a fastening strap which uses the trademark “Velcro” to secure to the heel. The support brace works by using a combination of removable cushion pads located in four strategically located areas of the support brace.

The first cushion pad is located at the bottom of the support brace which acts as a cushion for the heel. There are 2 cushion pads located on each side of the support brace to cushion each side of the heel. The last pad is located at the rear of the support brace to cushion the Achilles. With all cushion pads being removable you can isolate a given area.

The intent of this heel support brace is quite simple, to not only to cushion or pad the heel, but to provide superior support to all parts of the heel. As with many injuries sliding on a one piece support brace is very painful. However by using “Velcro” as a latch, it makes this support completely pain free and easy to put on. As with most injuries the external pads or cushions available rely on sticking on the pad or cushion directly to the skin. This support is designed to hold pads or cushions securely in place to prevent rubbing and irritation, also preventing any kind of reaction from the adhesive to the damaged area or areas. With all pads or cushions being removable this allows only the use of those pads or cushions that are needed.

### SUMMARY OF INVENTION

The support padded heel brace reduces pain and injury to the heel, Achilles and shin by reducing impact to said areas by using a series of pads. The brace also helps keep the natural form of the heel reducing natural time related injuries to the heel. It also helps with the rehabilitation of all facets of the heel. This brace is excellent for the athlete by giving the heel total and firm support. This brace will aid in sleeping as it reduces pain. The brace is perfect for the general public by reducing not only the typical causes of injury but assures comfort with all types of foot wear. It should be noted that this brace is not a fix all. It simply helps to prevent injury, aids with healing and adds comfort to the heel area.

### DETAILED DESCRIPTION OF THE DRAWINGS

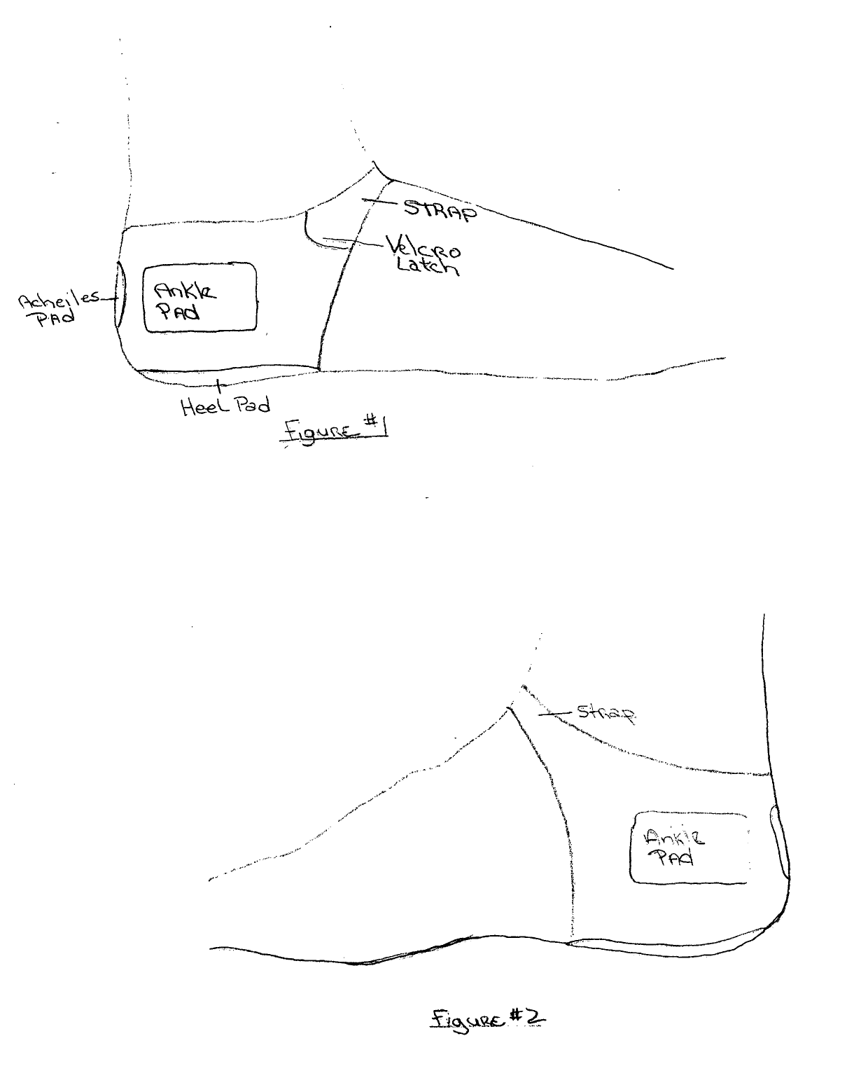

FIG. 1

This drawing is viewing at the support heel brace from the exterior of heel. It shows the “Velcro” latch. The outside pad and the bottom stitching of heel pad/cushion.

FIG. 2

This drawing is viewing the support heel brace from the exterior of heel. It shows the strap side. The outside pad and the bottom stitching of the heel pad/cushion.

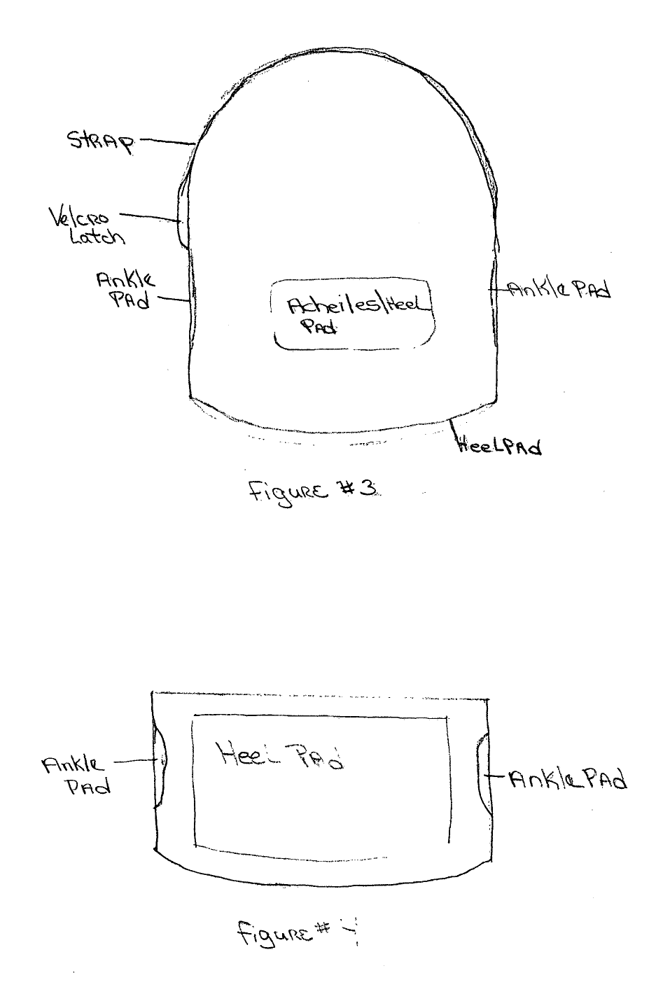

FIG. 3

This drawing is viewing the rear or back of the support heel brace. It shows the Achilles pad/cushion and the entry point of the heel pad/cushion.

FIG. 4

This drawing is viewing support heel brace from the interior of the bottom stitching of the heel pad/cushion.

### DETAILS

Each pad/cushion may vary in dimensions and thickness as follows:

Side Pad/Cushion:
Width varies from ¼″+/− to 1½″+/−.
Length varies from ¼″+/− to 1½″+/−.

Rear Pad/Cushion (Achilles):
Width varies from ¼″+/− to 1″+/−.
Length varies from ½″+/− to 4″+/−.

Bottom Pad/Cushion (Heel):
Width varies from ½″+/− to 4″+/−.
Length varies from 1″ +/− to 4″+/−.

Thickness (Pad/Cushion):
Thickness varies from 2/16″+/− to 1″+/−.

1. The heel support brace is a dynamic durable design which gives support and shape to the heel by using a series of pads. The brace ensures comfort to all parts of the heel. With this support you will never have a blister or soreness when breaking in new shoes. Also comforts and reduces the impact to the heel. Because of the braces design it will help the shape and natural integrity of heel prolonging the heel from breaking down hence reducing the natural causes of injuries to the heel. Because the brace is padded it also reduces the risk of heel spurs, chin splints and reduces the discomfort of the Achilles heel pain. Because of the flexibility of a removable pad system a variety of pads may be used. This will help make the heel support brace not only universal, as well as practicable but affordable to the consumer.
